(19) United States
(12) Patent Application Publication
(10) Pub. No.: US 2002/0147135 A1
(43) Pub. Date: Oct. 10, 2002

(54) Method and device for producing an adapted travel treatment plan for administering a medicine in the event of a long-haul journey

(76) Inventor: Oliver Schnell, Munchen (DE)

Correspondence Address: Thomas M. Champagne, IP Strategies, P.C., Suite 301, 806 7th Street N.W., Washington, DC, 20001, US

(21) Appl. No.: 10/034,196

(22) Filed: Dec. 20, 2001

(30) Foreign Application Priority Data
Dec. 21, 2000 (DE) 100 64 018.4
Dec. 21, 2000 (EP) 00128168.2

(51) Int. Cl. A61K 38/28; G06F 17/60

(52) U.S. Cl. 514/3; 705/3; 705/13

(57) ABSTRACT

Patients with certain chronic diseases such as diabetes require medical treatment according to a set time-related treatment plan. Such a treatment plan must be adapted accordingly in the case of long-haul journeys to countries with a time difference. A method for this has the steps of recording an regular treatment plan for administering the medicine, recording the point of departure and destination as well as the time of travel of the long-haul journey, determining the time zone difference between the point of departure and the destination and producing an adapted travel treatment plan based on the regular treatment plan depending on the time zone difference and the time of travel. The invention enables a patient to achieve a structured adaptation of his individual treatment plan to the time difference in the destination country of the journey in a convenient manner.

### TECHNICAL FIELD

Many illnesses such as diabetes, for example, call for regular treatment using certain medicines according to a treatment plan that is fixed in time. On long-haul journeys to countries with a time difference, these patients have to adapt the treatment plan, for example involving insulin and tablets that lower the blood sugar, accordingly. Only thus can sharp varations in blood sugar and potentially life-threatening derailments of the metabolism be avoided. The process of adaptation is geared to the respective therapy and is therefore directly dependent on the therapy plan or treatment plan, i.e. on the time and the number and structure of daily insulin injections and/or the blood-sugar-lowering tablets taken. Adherence to set application times is also necessary for hormonal contraceptives.

### PRIOR ART

Up to now, a treatment plan for treating a disease has been adapted for long-haul journeys without any structured or comprehensible concept. The result of this is that adaptation of the therapy is often not carried out properly, and uncertainty exists for the patient regarding the therapy each time a time difference occurs. This is a disadvantage in particular with reference to metabolism illnesses such as diabetes, in which the health of the patient depends essentially on administering therapeutics regularly.

### SUMMARY OF THE INVENTION

The invention makes it possible for the disadvantages of the prior art that have been described to be overcome and for the possibility to be opened up for the patient or the doctor responsible of adapting an existing treatment plan in a structured and comprehensible manner for a long-haul journey involving a time difference.

Preferred developments of the invention are described in the dependent claims.

The method according to the invention for producing an adapted travel treatment plan for administering a medicine in the event of a long-haul journey, which method can be executed on a computer system, has the following steps: recording of an regular treatment plan for administering the medicine, recording of the point of departure and destination and time of travel of the long-haul journey, determining the time zone difference between the point of departure and the destination, and producing an adapted travel treatment plan based on the regular treatment plan depending on the time zone difference and the time of travel. Regular treatment plan is taken here to mean the “normal” treatment plan prior to the long-haul journey or, following a transition period, after the journey. By computer system, a standalone computer, terminal device provided with a processor and storage device such as a mobile telephone or a sugar concentration measuring device or a networked system of several computers is meant.

The method according to the invention supplies the patient with a structured and comprehensible treatment plan for use on the long-haul journey. According to the time zone difference arising due to the long-haul journey and the respective time of travel, an individual treatment plan is produced for the patient based on the regular treatment plan. The patient receives clear instructions for therapy in a period following the changeover of the clock time necessitated by the long-haul journey.

To produce the travel treatment plan, a set of travel treatment plans is preferably determined depending on a non-application period between a last application according to the regular treatment plan, based on the local time at the point of departure of the long-haul journey, and the next application according to the regular treatment plan, based on the local time at the destination. The set of travel treatment plans is stored preferably in a storage device and the travel treatment plan to be applied in each case selected on the basis of the non-application period established.

The method according to the invention can be used for example to treat diabetics. In this case, travel treatment plans are preferably drawn up for all known insulin types and/or blood-sugar-lowering medicines that are licensed in a starting and/or destination country of the journey. It goes without saying in this regard that the treatment plans can be updated on the basis of newly licensed therapeutics or new medical discoveries. A further application is the administering of hormonal contraceptives. According to the invention, a treatment plan adapted to the long-haul journey is produced for the respective preparation, which plan facilitates reliable action of the preparation even on the journey.

Summertime (Daylight-saving time) adjustments are preferably also taken into consideration when calculating the time zone difference.

The point of departure and destination of the long-haul journey can be entered by the patient himself or determined via a satellite communications system. The method according to the invention can also be executed on a device that the patient carries with him. Alternatively, it is also possible for the patient to transfer the necessary information, such as the regular treatment plan and the travel information, via a communications link to a central computer that determines the individual treatment plan and makes this available for the patient to access via the Internet, for example. To obtain recommendations for treatment during long-haul journey, the device can also be used by others than patients, for example doctors or pharmacists. To safeguard privacy, password protection or the like can naturally be provided.

Depending on the illness to be treated, the travel treatment plan covers a fixed transition period following the time when the clock is changed that can be between less than 1 day and up to 14 days, for example. In the case of diabetes treatment, a period of 48 hours is preferred.

The device according to the invention for producing an adapted travel treatment plan for administering a medicine in the event of a long-haul journey comprises: a device for recording an regular treatment plan for administering the medicine, a device for drawing up a set of travel treatment plans based on the regular treatment plan for various time zone differences and Times of time zone changeover, a storage device for storing the set of adapted travel treatment plans, a device for recording the point of departure and destination of the long-haul journey, a device for determining the time zone difference, a selection device for selecting one of the stored travel treatment plans depending on the time zone difference and the time of time zone changeover, and an output device for outputting the selected treatment plan.

According to the invention, a method is proposed for administering a medicine calling for application according to a regular time-related treatment plan on a long-haul journey, comprising the steps: recording of the time-related treatment plan of the medicine, recording of the point of departure and destination and time of travel of the long-haul journey, determination of the time zone difference between the point of departure and the destination, production of an adapted travel treatment plan based on the time-related treatment plan depending on the time zone difference and the time of travel, and administering of the medicine in accordance with the adapted travel treatment plan.

### BRIEF DESCRIPTION OF DRAWINGS

The invention and further objects, features and advantages of this are explained in detail below with reference to preferred embodiments, with reference to the enclosed drawings, in which

FIG. 1 is a diagrammatic representation of an embodiment of a device according to the invention for producing a travel treatment plan;

FIG. 2 is a diagrammatic representation of a further embodiment of a device according to the invention for producing a travel treatment plan; and

FIG. 3 is a flow chart showing the method steps of the method according to the invention for producing an adapted travel treatment plan.

### DETAILED DESCRIPTION OF EMBODIMENTS

The invention is explained below in detail with reference to preferred embodiments.

FIG. 1 shows diagrammatically an embodiment of the device according to the invention for producing a travel treatment plan. The device is preferably kept compact in construction, so that the patient can carry it comfortably on journeys, and has an input device such as a keyboard, for example, for entering the patient's individual regular treatment plan (see description further below) and for entering the departure time and arrival time of the long-haul journey. On the basis of these data and the time of travel recorded, more precisely the time that the clock time is changed, the device 100 determines a modified travel treatment plan for adapting the therapy to the time changeover caused by the long-haul journey. The manner of calculation of the travel treatment plan is explained in detail later with reference to examples. An output device such as a screen or printer is used to output the travel treatment plan for the user/patient. It can also be provided that the device 100 emits optical or acoustic warning signals reminding the user that a treatment is due, for example using insulin. The device 100 can be integrated into a mobile terminal device, such as a laptop computer, an electronic organizer or a mobile telephone.

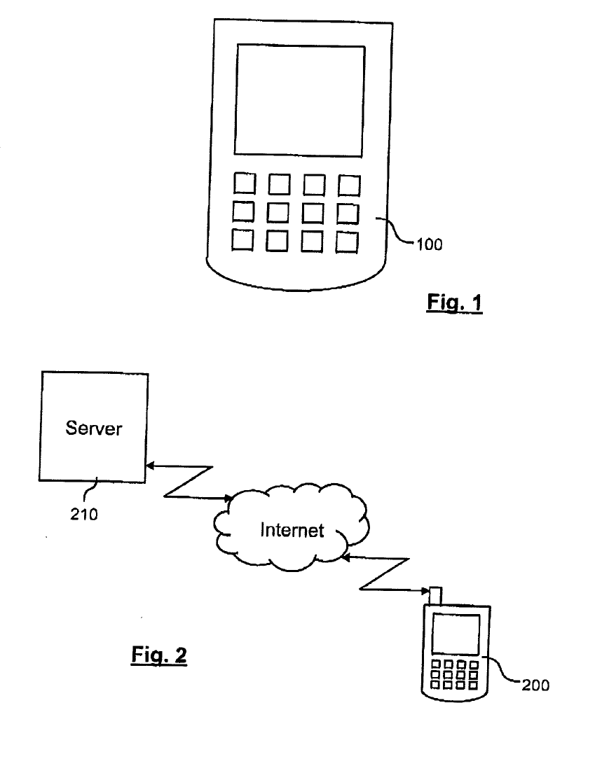

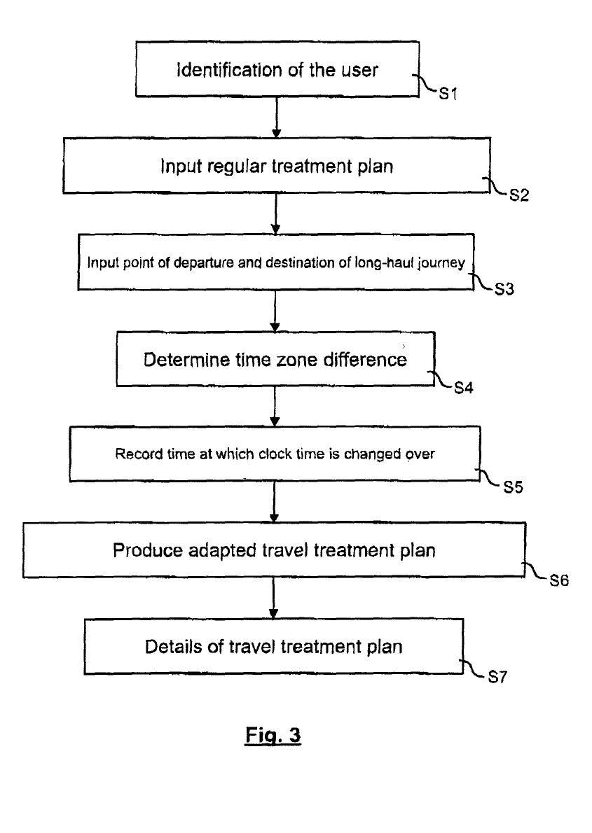

FIG. 2 shows a further embodiment of the device according to the invention. In this embodiment, a terminal device 200 with input and output devices is connected via a communications network such as the Internet to a server 210. The user here enters the travel data and his regular treatment plan, which data are then transmitted via the internet to the server computer 210. This then determines the pertinent individual travel treatment plan, which is then transmitted via the Internet to the user terminal device 200 for output. The embodiment in FIG. 2 has the advantage that adaptations to newly licensed medicines such as new insulin types can be undertaken more easily. It is also easily possible to adapt the modified travel treatment plans to the latest medical discoveries.

The method according to the invention can advantageously be integrated into a personal time planning program (Personal Information Manager, PIM), for example Microsoft Outlook or Lotus Notes. The travel treatment plan is then integrated into the personal, computer-aided time planning of the user.

Instead of entering the departure point and destination manually, it is also alternatively possible to carry out position determination of the terminal device 100 or 200 via a satellite navigation system (GPS). It is then sufficient for the user to enter his regular treatment plan once into the terminal device 200, which then checks the current location of the device (and of the user if the user is carrying it) on an ongoing basis and emits a warning signal as soon as the time difference has attained a value necessitating adaptation of the treatment plan. The user then only needs to press a button, whereupon the point at which the clock time is changed over is determined via an inbuilt clock and the adapted individual travel treatment plan is determined and output on the screen.

Alternatively, it is also possible to manage without a special user terminal device. The user can deposit his regular treatment plan on a server in a manner protected by password. As soon as he is travelling, he can access the information stored on the server from any computer with an Internet capability or the like via the Internet by entering his password and can enter the travel data, whereupon the adapted travel treatment plan is conveyed to the user by Internet.

FIG. 3 in the form of a flow chart shows the method steps for calculating the adapted travel treatment plan.

In step S1, the user identifies himself by a name, password etc.. Then he enters the regular treatment plan, i.e. the personal regular applications of medicine. In step S3, the point of departure and destination of the long-haul journey are entered by the user, which can be done in various ways, as explained above. Thereupon the method according to the invention determines the time zone difference in step S4 and also the time at which the clock is changed over (S5). Using this information and on the basis of the regular treatment plan entered, the adapted travel treatment plan is produced in method step S6, which plan is output in method step S7 via a screen, a printer or the like for the user.

The method for determining the adapted travel treatment plan is explained below in detail with reference to the practical example of diabetes therapy.

The invention is applicable to all departure times and points of departure of all time zones and to all arrival times and destinations of all time zones. The local times of the largest places with airports of all countries and time zones are stored. The local times are related for example to Greenwich Mean Time (Universal Time Coordinate (UTC)) and can be retrieved according to summer time and winter time and relative to the respective date.

The embodiment is designed for all forms and plans of current and future insulin therapies and is applicable to subcutaneous, intraperitoneal, intravenous and inhalative insulin therapy and to all other conceivable forms of treatment for diabetes, even those nor based on insulin It covers all treatment options involving tablets that lower blood sugar. All therapeutics licensed in a country are stored in a storage device (all insulins and other medicines that lower blood sugar).

The method is conceived so that in addition to the travel data the individual form of treatment is selected, the respective day-related doses of insulin or tablets is entered, and on the basis of a set of rules a recommendation is established for adaptation of the therapy depending on the direction and extent of the time difference for the time during the journey. The method thus contributes to optimising the metabolic situation of diabetics and prevents metabolism derailments by means of a proper, structured adaptation of the therapy.

In addition to the production of travel treatment plans for administering insulins and/or medicines that lower the blood sugar, the invention can also comprise recommendations for the intake of food, in particular of carbohydrates. The recommended amounts of carbohydrates are preferably indicated here in bread units (BU) and the amount of carbohydrates actually consumed entered by the user respectively.

The adapted treatment plan can also be used for any type and application of continuous blood-sugar-lowering therapy using insulin by means of insulin metering devices (insulin pumps). This form of treatment is based on a continuous application of insulin according to a programmed basal rate, which is oriented to the insulin requirement of the patient depending on the time of day (respectively x units per hour, e.g. 0.6 units/h from 0000-0300 hours (12 p.m.-3 a.m.), 1 unit/h from 0300-0800 hours (3 a.m.-8 a.m.), 0.8 unit/h from 0800-1400 hours (8 a.m.-2 p.m.), 0.7 unit/h from 1400-2000 hours ( 2 p.m.-8 p.m.), 0.8 unit/h from 2000-0000 hours (8.p.m.-12 p.m.). It is therefore determined individually and forms the daily recurring constant of the therapy. In addition, mealtime-related insulin is called up to cover the amount of carbohydrates supplied.

In the travel treatment plan, the basal rate is adapted in a structured manner to the time difference over a defined period (up to 10 days). Thus in the case of a time difference of 6 hours in an easterly direction, for example, the basal rate of insulin is advanced by one hour a day, until it is matched precisely to the new time after 6 days. In the case of a time difference of 12 hours in a westerly direction, it is retarded for example by 2 hours per day and is then matched after 6 days. It can also be retarded by 4 hours a day and be adapted after 3 days or by 6 hours a day and then be matched after 2 days. The degree of daily adaptation is variable and can be self-selected.

The mealtime-related insulin delivery (insulin-bolus) is regulated by the corresponding therapy plans of the travel treatment plan (e.g. insulin 1-1-1-0).

The method according to the invention can also include measurement of an actual blood sugar value of a user/patient and take the value thus ascertained into account for the treatment plan. The blood sugar value can be recorded separately by the patient using a separate device and then entered manually by the patient. However, it is also possible for the invention to be integrated into a device for measuring blood sugar. According to the invention, the blood sugar level can also be measured continuously by non-invasive methods or glucose sensors, it also being possible for indirect methods of measurement employing infrared beams for example to be used, which methods are based on the measurement of a sugar concentration in other body fluids.

Classes of Medicines Stored

All blood-sugar-lowering medicines licensed in Germany are stored. The data are updated continuously and updated as soon as new medicines are licensed. The medicines are classified according to their action profile and medicines with the same action profile are collected in one group in each case. The groups are stored relative to the therapy plans that they match and can be retrieved there.

Insulins

Insulins are classified according to their action profile and divided into five different groups of insulins (I1-I5) depending on the duration of the action. If new insulins should be licensed that cannot be classified in any of the groups mentioned, a new group is formed for storing these.

| | Group |
|---|---|
| 1 Short-acting human insulins | I1 |
| 2 Modified short-acting insulins | I2 |
| 3 Medium-acting, max. 12 h active mixed insulins | I3 |
| 4 Medium-acting, max. 12 h active basal insulins | I4 |
| 5 Medium-acting, max. 24 h active insulins | I5 |
| 6 Modified long-acting insulins | I6 |

However, groups with a duration of action other than those indicated can also be formed (longer or shorter duration of action than I1-I6).

Blood-Sugar-Lowering Tablets

Blood-sugar-lowering tablets (oral antidiabetics) are summarized into 8 groups according to their active mechanism. If new blood-sugar-lowering medicines should be licensed that cannot be classified into any of the groups mentioned, a new group is formed for storing these.

| | Group |
|---|---|
| 1 Alpha-glucosidase-inhibitors | T1 |
| 2 Metformin | T2 |
| 3 Glibenclamide | T3 |
| 4 Tolbutamide | T4 |
| 5 Glimepiride | T5 |
| 6 Other sulphonylurea derivates | T6 |
| 7 Prandial glucose regulators | T7 |
| 8 Glitazone | T8 |

Therapy Plans

All treatment plans for diabetics are stored in a storage device. The related groups of medicines are assigned to each treatment plan. The plans are based on the fact that insulins are injected at set times and blood-sugar-lowering tablets are taken at set times, and take account of the fact that an identical scheme of action exists within the groups of insulins and blood-sugar-lowering tablets.

Insulin Therapy Plans

Insulin is injected at set times (morning, lunchtime, evening, late). Linked to this is the intake of meals (morning, lunchtime, evening, late no meal).

| Period | Time | Abb. insulin | Meal | Abb. meal |
|---|---|---|---|---|
| Morning | 7 | IF | Breakfast | F |
| Lunchtime | 13 | IM | Lunch | M |
| Evening | 19 | IA | Dinner | A |
| Late | 22 | IS | No or | — |
| | | | small | |
| | | | regular | |
| | | | meal | |

Since diabetics do not always inject insulin at exactly these times, a period is specified for insulin application from which the diabetic can select his individual time for an insulin injection: morning 0600-0800 hrs, lunchtime 1200-1400 hrs, evening 1800-2000 hrs, late 2100-2300 has. To obtain a constant time for the calculation, the insulin injection is related to the times set in the plan (0700, 1300, 1900, 2200 hrs) (7 a.m., 1 p.m., 7 p.m., 10 p.m.). The system, however, applies to all other times.

The method according to the invention calculates any additional insulin injections that should become necessary and may be linked to any additional interim meals.

| Period | Time | Abb. insulin | Meal | Abb. meal |
|---|---|---|---|---|
| Variable | Variable | IZ | Interim meal | Z |

Insulin Plans

The times are defined via the states 0 (no) and 1 (yes):

Examples: insulin morning, lunchtime, evening, late

1-1-1-1

Insulin morning, no insulin lunchtime, insulin evening, no insulin late

1-0-1-0

Suitable insulin groups are stored for each insulin therapy plan. Each insulin therapy plan acquires different versions due To the storage of various insulin groups. A set of rules is defined for each version of each insulin plan for all time differences in an easterly and westerly direction, from which therapy alterations are calculated in a structured manner for the time difference.

Combinations of the same or different insulin plans and the same or different versions with the same or different insulin groups stored are possible. All insulin plans are integrated with all versions.

Insulin 1× Daily

Possible times; morning, lunchtime, evening, late

| Version | Insulin | Insulin groups stored |
|---|---|---|
| a | 1-0-0-0 | I1,I2,I3,I4,I5,I6 |
| b | 0-1-0-0 | I1,I2,I3,I4,I5,I6 |
| c | 0-0-1-0 | I1,I2,I3,I4,I5,I6 |
| d | 0-0-0-1 | I1,I2,I3,I4,I5,I6 |

Insulin 2× Daily

Possible times: morning, lunchtime, evening, late

| Version | Insulin | Insulin groups stored |
|---|---|---|
| a | 1-1-0-0 | I1,I2,I3,I4,I5,I6 |
| b | 1-0-1-0 | I1,I2,I3,I4,I5,I6 |
| c | 1-0-0-1 | I1,I2,I3,I4,I5,I6 |
| d | 0-1-0-1 | I1,I2,I3,I4,I5,I6 |
| e | 0-0-1-1 | I1,I2,I3,I4,I5,I6 |
| f | 0-1-1-0 | I1,I2,I3,I4,I5,I6 |

Insulin 3× Daily

Possible times: morning, lunchtime, evening, late

| Version | Insulin | Insulin groups stored |
|---|---|---|
| a | 1-1-1-0 | I1,I2,I3,I4,I5,I6 |
| b | 1-0-1-1 | I1,I2,I3,I4,I5,I6 |
| c | 0-1-1-1 | I1,I2,I3,I4,I5,I6 |
| d | 1-1-0-1 | I1,I2,I3,I4,I5,I6 |

Insulin 4× Daily

Possible times: morning, lunchtime, evening, late

| Version | Insulin | Insulin groups stored |
|---|---|---|
| a | 1-1-1-1 | I1,I2,I3,I4,I5,I6 |

Insulin>4× Daily

Possible times: morning, lunchtime, evening, late and in addition all other times

Insulin Groups Stored I1, I2, I3, I4, I5,I6

Insulin Pump Therapy

Insulin Groups Stored I1, I12

Therapy plans involving blood-sugar-lowering tablets

Tablets are taken at set times (morning, lunchtime, evening, late). Linked to this is the intake of meals (morning, lunchtime, evening, late no meal).

| Period | Time | Abb. Tablet | Meal | Abb. meal |
|---|---|---|---|---|
| Morning | 7 | TF | Breakfast | F |
| Lunchtime | 13 | TM | Lunch | M |
| Evening | 19 | TA | Dinner | A |
| Late | 22 | TS | No or | — |
| | | | small | |
| | | | regular | |
| | | | meal | |

Since diabetics do not always take tablets at exactly these times, a period is specified for taking them from which the diabetic can select his individual time for taking them: morning 0600-0800 hrs, lunchtime 1200-1400 hrs, evening 1800-2000 hrs, late 2100-2300 hrs. To obtain a constant time for the calculation, the intake is referred to the times set in the plan (0700, 1300, 1900, 2200 hrs) (7 a.m., 1 p.m., 7 p.m., 10 p.m.).

The method according to the invention calculates any additional tablet intakes that should become necessary and may be linked to any additional interim meals.

| Period | Time | Abb. Tablet | Meal | Abb. meal |
|---|---|---|---|---|
| Variable | Variable | TZ | Interim meal | Z |

Tablet Therapy Plans

The times are defined via the states 0 (no) and 1 (yes):

Examples: tablet morning, lunchtime, evening, late

1-1-1-1

Tablet morning, no tablet lunchtime, tablet evening, no tablet late

1-0-1-0

Suitable tablet groups are stored for each therapy plan involving blood-sugar-lowering tablets. Each therapy plan involving blood-sugar-lowering tablets acquires different versions due to the storage of various groups of tablets. A set of rules is defined for each version of each therapy plan involving blood-sugar-lowering tablets for all time differences in an easterly and westerly direction, from which therapy alterations are calculated in a structured manner for the time difference.

All combinations (tablet 1 4× daily) of versions are possible in the same way as for insulin therapy when taking various blood-sugar-lowering tablets.

Adaptation of diabetes therapy in the event of a time difference

Time axes for all time differences are stored in an data base (time progression) for each treatment plan and for each group of medicines stored. A worksheet contains a Time axis of time up to now “time old” in one line and on lines below this, related to “time old”, all possible time axes of the time difference (“time new”).

A dedicated set of rules is created in a further data base for each treatment plan and each group of medicines stored. This set of rules is dependent on the frequency of intake and the respective group of medicines as well as on the dose up to now related to the time of day.

A set of rules related to the group of medicines is necessary, as every group of medicines with the related medicines has a definite action profile that is characteristic of it, calling for a set of rules.

Calculating adaptation of the treatment in the event of a time difference is based on the following information:

a) The departure time and point of departure and the arrival time and destination are each entered with the date. The program calculates the time difference in hours from this data along with its direction (1 h-12 h, westerly or easterly).

b) The treatment plan or plans to date are entered. The previous time or times of intake are established thereby.

c) The previous medicine or medicines are selected relative to the treatment plan or plans.

d) The previous insulin dose or doses in units of insulin (IU, U) and/or the previous number of tablets is entered.

From information a)-d) the related data base of the time progression is selected and the data entered are integrated into this. The time progression worksheet serves as an aid to determining the related set of rules. Calculation of the set of rules via the time progression worksheet is based on the following procedure:

a) The time at which the clock time is changed over is critical for the calculation. Normally this is the time of arrival, meaning that during the flight “time old” applies with the corresponding plan. At the time of arrival, depending on the place, i.e. depending on the time difference in the Excel worksheet, the corresponding “time new” is invoked. The previous treatment plan is stored at this, but just displaced in Time. The option also exists of planning to change over the time during the flight. This does not change anything with regard to the procedure for calculation.

b) To calculate the adaptation, the following reference is created: the time in hours between the last intake of medicine on time axis “time old” and the envisaged next intake of medicine on time axis “time new” is calculated. This is composed of:

Arrival time (when changing time over on arrival) minus time of last medicine intake=x (h)

Time of next envisaged medicine intake of the same group of medication minus arrival time (when changing time over on arrival)=y (h)

x (h) plus y (h)=period (h) between last and next envisaged intake of medicine

Adaptation of the therapy results from the stored set of rules from the period (h) between the last and next envisaged medicine intake. For every period (h) between) the last and next envisaged medicine intake of the same group of medication possible with regard to the corresponding treatment plan, a specific rule applies that is clear in the set of rules. This is directly dependent on the period (h) calculated between the last and the next envisaged medicine intake.

According to the rule stored, a treatment recommendation is given for the at least following 24 h following the time of arrival. This can consist in the treatment a) being continued unchanged, b) the dose of medicine intake being changed and the time and meal remaining the same, c) an envisaged medicine intake and meal are left out, d) an additional medicine intake with an unchanged dose and an additional meal are taken or e) an additional medicine intake with a change in dose and an additional meal are taken. Combinations of a, b, c, d and e are possible.

The invention thus makes it possible To produce a structured and comprehensible treatment plan for the patient that the latter can retrieve and apply easily. Uncertainties regarding the correct therapy can thus be avoided on a long-haul journey and the patient's quality of life improved considerably.

The invention is not restricted to use for treating diabetes. Other application examples are the treatment of blood pressure illnesses (hypertonia, hypotonia) or the regular administering of blood-thinning therapeutics such as marcumar or acetyl salicylic acid or, as described, the administering of hormonal contraceptives.

Appendices 1 to 3 contain examples of travel treatment plans for flights Munich—New York, Frankfurt—Tokyo and Mexico—London Heathrow.

[image]

[image]

[image]

[image]

[image]

[image]

[image]

[image]

[image]

[image]

[image]

[image]

1. Method executable on a computer system for producing an adapted travel treatment plan for administering a medicine in the event of a long-haul journey, having the steps:
Recording of an regular treatment plan for administering the medicine,
recording of the point of departure and destination as well as the time of travel of the long-haul journey,
determining the time zone difference between the point of departure and destination, and
producing an adapted travel treatment plan based on the regular treatment plan depending on the time zone difference and the time of travel.

2. Method according to claim 1, a set of travel treatment plans being drawn up depending on a non-application period between a last application according to the regular treatment plan, taking the local time at the point of departure of the long-haul journey as a basis, and the next application according to the regular plan, taking the local time at the destination as a basis.

3. Method according to claim 2, the set of travel treatment plans being stored in a storage device, the non-application time being determined on the basis of the recorded point of departure and destination and the time of travel or time of time zone changeover and the travel treatment plan to be applied being selected from the set of travel treatment plans on the basis of the non-application period.

4. Method according to claim 3, various travel treatment plans being produced for various types of insulin and/or blood-sugar-lowering medicines.

5. Method according to claim 4, a travel treatment plan containing additional notes on recommended times for food intake, in particular of carbohydrates.

6. Method according to claim 5, also comprising recording of an actual intake of carbohydrates due to supplying of food.

7. Method according to claim 6, the carbohydrate intake being recorded by entry by the user.

8. Method according to claim 4, also comprising recording of the blood sugar concentration of the user.

9. Method according to claim 4, also comprising continuous recording of the blood sugar concentration by glucose sensors or non-invasive techniques.

10. Method according to claim 8, also comprising continuous recording of the sugar concentration in other body fluids.

11. Method according to claim 4, the various insulin types being classified according to their action profile.

12. Method according to claim 4, all insulin and/or blood-sugar-lowering therapeutics licensed in a starting and/or destination country of a journey being included in the set of travel treatment plans.

13. Method according to claim 12, the set of travel treatment plans being updated in the case of newly licensed insulin preparations and/or blood-sugar-lowering therapeutics.

14. Method according to claim 4, the set of travel treatment plans being updated in line with new medical findings.

15. Method according to claim 1, any local time arrangements such as summertime adjustments in individual time zones being taken into account when producing the travel treatment plan.

16. Method according to claim 1, the point of departure and destination of the long-haul journey being ascertained via a satellite communications system (GPS)

17. Method according to claim 1, the travel treatment plan produced being retrievable from a central computer via the Internet and/or wireless communication media.

18. Method according to claim 1, the travel treatment plan covering a set transition period following the time at which the clock time is changed over.

19. Method according to claim 18, the transition period being up to 14 days, preferably roughly 48 hours.

20. Method according to claim 3, a related set of travel treatment plans being stored for a preset regular treatment plan as a worksheet of a spreadsheet program.

21. Method according to claim 20, one line containing the time sequence of the regular treatment plan and following lines containing the related travel treatment plan for various time zone differences.

22. Method according to claim 1, a travel treatment plan being produced for continuous blood-sugar-lowering therapy by means of an insulin dosing device.

23. Method according to claim 1, a travel treatment plan being produced for a contraceptive.

24. Device for producing an adapted travel treatment plan for administering a medicine in the event of a long-haul journey, comprising:
a device for recording an regular treatment plan for administering the medicine,
a device for producing a set of travel treatment plans based on the regular treatment plan for various time zone differences and times of time zone changeover,
a storage device for storing the set of adapted travel treatment plans,
a device for recording the point of departure and destination of the long-haul journey,
a device for determining the time zone difference,
a selection device for selecting one of the stored travel treatment plans depending on the time zone difference and the time of time zone changeover, and
an output device for outputting the selected treatment plan.

25. Device according to claim 24, the device for producing a set of travel treatment plans determining a non-application period from the last application according to the regular treatment plan according to the starting time zone to the next application according to the treatment plan according to the destination time zone as an ordering parameter for the set of travel treatment plans.

26. Device according to claim 24, the travel treatment plan produced being provided for the treatment of diabetes.

27. Device according to claim 26, the treatment plan comprising insulin doses, blood-sugar-lowering therapeutics and/or instructions for the intake of meals.

28. Device according to claim 24, the storage device containing sets of travel treatment plans for all licensed insulin types and/or blood-sugar-lowering therapeutics licensed in the country of departure and/or destination.

29. Device according to claim 24, the device producing an adapted travel treatment plan for a continuous blood-sugar-lowering therapy by means of an insulin dosing device.

30. Device according to claim 24, the device for calculating the time zone difference taking local time adjustments such as summertime adjustments into account.

31. Device according to claim 24, also having an input device for entering the point of departure and destination of the long-haul journey.

32. Device according to claim 24, also having a device for determining position via a satellite communications system (GPS).

33. Device according to claim 24, also having an acoustic or optical warning device to give a reminder of a required application of a medicine.

34. Device according to claim 24, also having a display device to display the travel treatment plan.

35. Device according to claim 24, the device being integrated into a mobile terminal device such as a laptop computer, an electronic organizer (Personal Digital Assistant, PDA) or a mobile telephone.

36. Device according to claim 24, the device being integrated into an apparatus for measuring the blood sugar values of a user.

37. Device according to claim 24, the device being integrated into an apparatus for the continuous measurement of the sugar concentration of a user.

38. Computer program product with program code for the production, on a computer, of an adapted travel treatment plan for administering a medicine in the event of a long-haul journey, having the steps:
recording of an regular treatment plan for administering the medicine,
recording of the point of departure and destination as well as the time of travel of the long-haul journey,
determining the time zone difference between the point of departure and the destination, and
producing an adapted travel treatment plan based on the regular treatment plan depending on the time zone difference and the time of travel.

39. Storage medium with stored computer program for producing an adapted travel treatment plan for administering a medicine in the event of a long-haul journey by means of a computer due to the following steps:
recording of an regular treatment plan for administering the medicine,
recording of the point of departure and destination as well as the time of travel of the long-haul journey,
determining the time zone difference between the point of departure and the destination, and
producing an adapted travel treatment plan based on the regular treatment plan depending on the time zone difference and the time of travel.

40. Method for administering a medicine calling for application according to a regular time-related treatment plan, on a long-haul journey, having the steps:
recording of the time-related treatment plan of the medicine,
recording of the point of departure and destination as well as the time of travel of the long-haul journey,
determining the time zone difference between the point of departure and the destination,
producing an adapted travel treatment plan based on the time-related treatment plan depending on the time zone difference and the time of travel, and
administering the medicine according to the adapted travel treatment plan.

41. Method according to claim 40 for administering insulin preparations and/or blood-sugar-lowering media.

42. Method according to claim 40 for administering contraceptives.
